(19) United States
(12) Patent Application Publication
(10) Pub. No.: US 2026/0083771 A1
(43) Pub. Date: Mar. 26, 2026

(54) Biopolymers for Ophthalmic Use

(71) Applicant: ExoPolymer, Inc., Mountain View, CA (US)

(72) Inventor: Derek Wells, Palo Alto, CA (US)

(73) Assignee: ExoPolymer, Inc., Mountain View, CA (US)

(21) Appl. No.: 19/109,469

(22) PCT Filed: Sep. 13, 2023
(86) PCT No.: PCT/US2023/074059

§ 371 Date: Mar. 6, 2025

Related U.S. Application Data
(60) Provisional application No. 63/406,552, filed on Sep. 14, 2022.

(51) Int. Cl. A61K 31/715; A61K 9/00; A61P 27/04

(52) U.S. Cl. CPC A61K 31/715; A61K 9/0048; A61P 27/04

(57) ABSTRACT

Provided herein are biopolymers comprising repeating polysaccharide units, preparations of biopolymers, and ophthalmic compositions comprising biopolymers, as well as methods of use.

### CROSS-REFERENCE TO RELATED APPLICATIONS

This application claims the benefit of priority of U.S. Provisional Application No. 63/406,552, filed Sep. 14, 2022, which is incorporated by reference herein in its entirety for any purpose.

### FIELD

Provided herein are biopolymers comprising repeating polysaccharide units, preparations of biopolymers, and ophthalmic compositions comprising biopolymers, as well as methods of use.

### BACKGROUND

Keratoconjunctivitis sicca or dry eye disease (DED) affects millions of people and can be caused by factors including aging, side effects of medication, and illness. There is an increased prevalence of DED in those over the age of 50 (Farrand et al., 2017), and a strong negative impact of DED on quality of life, with studies showing that the effects of dry eye on daily living are comparable to severe angina (Buchholz et al., 2006; Schiffman et al., 2003).

Many treatments are available for DED, from simple OTC eye drops containing petrochemical-derived ingredients such as glycerol, polysorbates, or polyethylene glycol, to prescription medications that contain immunosuppressants such as cyclosporine. OTC products such as TheraTears® (Akorn) and Refresh@(Allergan) contain carboxymethyl cellulose (CMC) and provide temporary relief but must be used multiple times throughout the day for maximum effect. Prescription treatments such as Xiidra® (Shire) and Restasis® (Allergan) are effective but require continued use and can be cost prohibitive. The study of DED and its possible treatment have driven substantial academic and industrial research. For example, recent studies have focused on new combinations of existing polymers (Simmons and Vehige, 2017), and major pharmaceutical companies have pursued new, prescription-based glycoprotein products such as lubricin (Lubris BioPharma/Novartis). Materials that provide increased moisture, lubrication, and have longer residence times on the surface of the eye are highly sought after in the treatment of ocular diseases such as DED. Another important attribute of polymer ingredients used in artificial tear formulations is shear thinning behavior. Recent studies have shown that polymers that are less viscous at high shear rates and more viscous at low shear rates (non-Newtonian behavior) provide benefits over other ingredients (Arshinoff 2021). These types of polymers thus spread more effectively during blinking, yet provide improved coverage and hydration when the eye is open.

There are multiple products for the treatment of DED that include the biopolymer hyaluronic acid (HA) as the active ingredient, due to its exceptional ability to bind water and viscoelastic properties. Products such as Hydrasense® (Bayer) and I-Drop® Pur Gel (I-MED Pharma) may contain up to 0.3% (w/v) HA and may provide benefits such as longer efficacy duration and fewer applications. A recent survey of several studies has shown that HA may increase tear formation as well as other factors associated with DED in comparison to either saline or common artificial tear formulations (Yang 2021).

HA may be effective as a DED treatment since it is a natural component of the eye vitreous, although it is not found naturally in tears. Although HA is a common ingredient in many formulations, one study showed that patients actually preferred a CMC-based formulation over HA in eye drops (Baudouin et al., 2012). Further, HA found in products like those mentioned above is typically animal-derived as a secondary product from porcine or bovine processing facilities. It can also be produced by fermentation of Streptococcus zooepidemicus or related species, pathogenic bacteria that naturally incorporate HA into their cellular capsule. As consumer trends toward clean-label, animal- and cruelty-free products become more commonplace, the inclusion of HA as a functional ingredient may pose challenges to formulators and suppliers.

There have been relatively few efforts to identify novel polymers for DED treatment, or to generate new products that are highly effective and more widely available. ExoPolymer can produce a portfolio of high moisture-binding biopolymers that are highly efficacious in the treatment of DED. An alternative product that shows improved function, reduces the number of applications per day, can remain on the ocular surface longer, displays favorable shear-thinning properties, and can be produced at substantially lower cost, would allow for substantial market growth and expansion. There is demand for new, high-functioning moisture binding carbohydrate-based polymers to address a range of needs for ophthalmic conditions.

### SUMMARY

The present disclosure provides naturally produced, non-animal-derived carbohydrate biopolymers having improved efficacy in the treatment of ocular conditions, including dry eye and dry eye disease (DED), as well as their use in ocular conditions. Biopolymers of the present disclosure are particularly suited to ophthalmic applications, and lack cytotoxicity in standardized testing. Furthermore, the biopolymers of the present disclosure, when used as functional ingredients in standard ophthalmic formulations, show improved efficacy in the treatment of ocular conditions, including dry eye and dry eye disease (DED).

Biopolymers of the present disclosure are improved over agents such as HA, at least because they are not derived from animal sources and can be made through fermentation of non-pathogenic microbes using agricultural feedstocks.
Embodiment 1. An ophthalmic composition comprising a biopolymer preparation comprising a biopolymer that is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated.
Embodiment 2. The ophthalmic composition of embodiment 1, wherein the glucose and galactose are linked by 3-1,3 glycosidic bonds and α-1,3 glycosidic bonds.
Embodiment 3. An ophthalmic composition comprising a biopolymer preparation comprising a biopolymer that is composed of repeating disaccharide units of the structure:

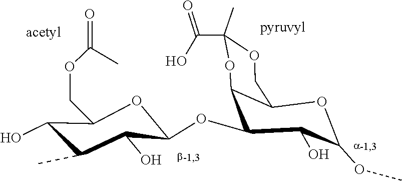

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety.
Embodiment 4. The ophthalmic composition of any one of embodiments 1-3, wherein the molar ratio of glucose:galactose:pyruvyl:acetyl in the biopolymer is 1:1:0.4-1:0.6-1.
Embodiment 5. The ophthalmic composition of any one of embodiments 1-4, wherein the average molecular weight of the biopolymer in the biopolymer preparation is less than 3,000 kDa, less than 1,000 kDa, less than 300 kDa, less than 100 kDa, or less than 40 kDa.
Embodiment 6. The ophthalmic composition of any one of embodiments 1-4, wherein the average molecular weight of the biopolymer in the biopolymer preparation is 0.5 kDa to 40 kDa.
Embodiment 7. The ophthalmic composition of any one of the preceding embodiments, wherein the ophthalmic composition comprises about 0.05 to about 5% w/v biopolymer.
Embodiment 8. The ophthalmic composition of any one of embodiments 1-6, wherein the ophthalmic composition comprises about 0.5 to about 2% w/v biopolymer.
Embodiment 9. The ophthalmic composition of any one of the preceding embodiments, wherein the biopolymer preparation is capable of absorbing at least the same amount, at least 1.5-fold, at least 2-fold, or at least 3-fold more water than an equal amount of hyaluronic acid.
Embodiment 10. The ophthalmic composition of any one of the preceding embodiments, wherein the biopolymer preparation is capable of absorbing an amount of water that is at least 100%, at least 200%, at least 300%, at least 400%, or at least 500% the initial dry weight of the biopolymer preparation.
Embodiment 11. The ophthalmic composition of embodiment 9 or embodiment 10, wherein water absorption is measured by placing a dry sample of the biopolymer preparation in a humidified chamber at 30° C. for five days.
Embodiment 12. The ophthalmic composition of any one of the preceding embodiments, wherein the biopolymer preparation is less than 25%, less than 20%, less than 15%, less than 10%, less than 5%, less than 3%, or less than 1% succinoglycan, or is substantially free of succinoglycan.
Embodiment 13. The ophthalmic composition of any one of the preceding embodiments, wherein the ophthalmic composition is a sterile fluid.
Embodiment 14. An ophthalmic composition comprising a biopolymer preparation comprising a biopolymer that is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated.
Embodiment 15. The ophthalmic composition of embodiment 14, wherein the glucose and galactose are linked by β-1,3 glycosidic bonds and α-1,3 glycosidic bonds.
Embodiment 16. An ophthalmic composition comprising a biopolymer preparation, wherein the biopolymer is composed of repeating disaccharide units of the structure:

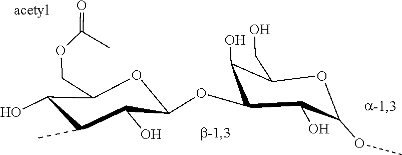

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated.
Embodiment 17. The ophthalmic composition of any one of embodiments 14-16, wherein the molar ratio of glucose:galactose:pyruvyl:acetyl in the biopolymer is 1:1:<0.5:0.6-1.
Embodiment 18. The ophthalmic composition of any one of embodiments 14-17, wherein the average molecular weight of the biopolymer in the biopolymer preparation is less than 3,000 kDa, less than 1,000 kDa, less than 300 kDa, less than 100 kDa, or less than 40 kDa.
Embodiment 19. The ophthalmic composition of any one of embodiments 14-17, wherein the average molecular weight of the biopolymer in the biopolymer preparation is 0.5 kDa to 40 kDa.
Embodiment 20. The ophthalmic composition of any one of embodiments 14-19, wherein the ophthalmic composition comprises about 0.05 to about 5% w/v biopolymer.
Embodiment 21. The ophthalmic composition of any one of embodiments 14-19, wherein the ophthalmic composition comprises about 0.5 to about 2% w/v biopolymer.
Embodiment 22. The ophthalmic composition of any one of embodiments 14-21, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.
Embodiment 23. The ophthalmic composition of any one of embodiments 14-22, wherein the biopolymer preparation is capable absorbing an amount of water that is at least 100%, at least 200%, at least 300%, or at least 400% the initial dry weight of the biopolymer preparation.
Embodiment 24. The ophthalmic composition of embodiment 22 or embodiment 23, wherein water absorption is measured by placing a dry sample of the biopolymer preparation in a humidified chamber at 30° C. for five days.
Embodiment 25. The ophthalmic composition of any one of embodiments 14-24, wherein the biopolymer preparation is less than 25%, less than 20%, less than 15%, less than 10%, less than 5%, less than 3%, or less than 1% succinoglycan.
Embodiment 26. The ophthalmic composition of any one of embodiments 14-25, wherein the ophthalmic composition is a sterile fluid.
Embodiment 27. An ophthalmic composition comprising a biopolymer preparation comprising a biopolymer that is composed of repeating polysaccharide units of the structure:

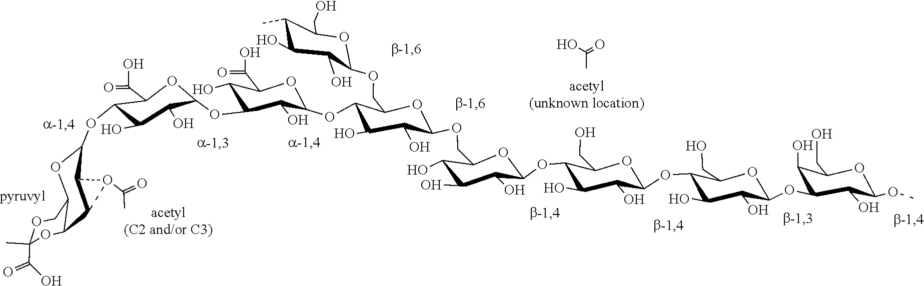

wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 1% of the polysaccharide units lack the pyruvyl moiety.
Embodiment 28. The ophthalmic composition of embodiment 27, wherein the average molecular weight of the biopolymer in the biopolymer preparation is less than 3,000 kDa, less than 1,000 kDa, less than 300 kDa, less than 100 kDa, or less than 40 kDa.
Embodiment 29. The ophthalmic composition of embodiment 27, wherein the average molecular weight of the biopolymer in the biopolymer preparation is 1.6 kDa to 40 kDa.
Embodiment 30. The ophthalmic composition of any one of embodiments 27-29, wherein the ophthalmic composition comprises about 0.05 to about 5% w/v biopolymer.
Embodiment 31. The ophthalmic composition of any one of embodiments 27-29, wherein the ophthalmic composition comprises about 0.5 to about 2% w/v biopolymer.
Embodiment 32. The ophthalmic composition of any one of embodiments 27-31, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.
Embodiment 33. The ophthalmic composition of any one of embodiments 27-32, wherein the biopolymer preparation is capable of absorbing an amount of water that is at least 100%, at least 200%, at least 300%, or at least 400% the initial dry weight of the biopolymer preparation.
Embodiment 34. The ophthalmic composition of embodiment 32 or embodiment 33, wherein water absorption is measured by placing a dry sample of the biopolymer preparation in a humidified chamber at 30° C. for five days.
Embodiment 35. The ophthalmic composition of any one of embodiments 27-33, wherein the ophthalmic composition is a sterile fluid.
Embodiment 36. An ophthalmic composition comprising a biopolymer preparation comprising a biopolymer that is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45.
Embodiment 37. The ophthalmic composition of embodiment 36, wherein the polysaccharide unit comprises at least one galactose linked to at least one glucose.
Embodiment 38. The ophthalmic composition of embodiment 37, wherein at least one galactose is linked to a glucose through a β-1,3 glycosidic bond.
Embodiment 39. The ophthalmic composition of any one of embodiments 36-38, wherein the average molecular weight of the biopolymer in the biopolymer preparation is less than 3,000 kDa, less than 1,000 kDa, less than 300 kDa, less than 100 kDa, or less than 40 kDa.
Embodiment 40. The ophthalmic composition of any one of embodiments 36-38, wherein the average molecular weight of the biopolymer in the biopolymer preparation is 0.5 kDa to 40 kDa.
Embodiment 41. The ophthalmic composition of any one of embodiments 36-40, wherein the ophthalmic composition comprises about 0.05 to about 5% w/v biopolymer.
Embodiment 42. The ophthalmic composition of any one of embodiments 36-40, wherein the ophthalmic composition comprises about 0.5 to about 2% w/v biopolymer.
Embodiment 43. The ophthalmic composition of any one of embodiments 36-42, wherein the biopolymer preparation is capable of absorbing at least the same amount, at least 1.1-fold, at least 1.5-fold, at least 2-fold, or at least 3-fold more water than an equal amount of hyaluronic acid.
Embodiment 44. The ophthalmic composition of any one of embodiments 36-43, wherein the biopolymer preparation is capable of absorbing an amount of water that is at least 100%, at least 200%, at least 300%, or at least 400% the initial dry weight of the biopolymer preparation.
Embodiment 45. The ophthalmic composition of embodiment 43 or embodiment 44, wherein water absorption is measured by placing a dry sample of the biopolymer preparation in a humidified chamber at 30° C. for five days.
Embodiment 46. The ophthalmic composition of any one of embodiments 36-45, wherein the ophthalmic composition is a sterile fluid.
Embodiment 47. The ophthalmic composition of any one of the preceding embodiments, wherein the composition comprises hyaluronic acid.
Embodiment 48. The ophthalmic composition of any one of embodiment 1-46, wherein the composition does not comprise hyaluronic acid.
Embodiment 49. The ophthalmic composition of any one of the preceding embodiments, wherein the composition is a fluid.
Embodiment 50. The ophthalmic composition of any one of the preceding embodiments, wherein the ophthalmic composition at least 70%, at least 80%, at least 85%, at least 90%, or at least 95% water.
Embodiment 51. A method of treating or preventing an ocular condition in a subject, comprising administering a therapeutically effective amount of the ophthalmic composition of any one of the preceding claims to the eye of the subject.
Embodiment 52. The method of embodiment 51, wherein the ocular condition is dry eye disease (DED).
Embodiment 53. The method of embodiment 51 or embodiment 52, where the ophthalmic composition is an ophthalmic oil-in-water emulsion, eye hydrogel, eye drop solution, eyebath, eye lotion, eye insert, eye ointment, eye foam, or eye spray.
Embodiment 54. The method of any one of embodiments 51-53, wherein the ophthalmic composition is an eye drop solution.
Embodiment 55. The method of any one of embodiments 51-54, wherein the ophthalmic composition is administered prophylactically.
Embodiment 56. The method any one of embodiments 51-54, wherein the ophthalmic composition is administered therapeutically after the onset of ocular disease.
Embodiment 57. The method of any one of embodiments 51-56, wherein the ophthalmic composition results in improvement or alleviation of one or more symptoms selected from dryness, burning, ocular itching, ocular discomfort, photophobia, foreign body sensation, blurry vision, grittiness, scratchiness, graininess, and visual disturbance and/or loss, including blurred vision, reduced reading speed, and loss in visual acuity.

### BRIEF DESCRIPTION OF THE DRAWINGS

FIG. 1. Representative repeating unit of a polysaccharide biopolymer. Individual sugar residues are linked by glycosidic bonds. Polysaccharide-based biopolymers consist of a main chain of sugar residues that are connected by glycosidic linkages. The main chain may also have a side chain consisting of sugar residues, and each may have different chemical modifications. The main and side chains of a biopolymer, along with chemical modifications, make up a repeating unit. Repeating units are connected by glycosidic linkages to generate longer polymers.

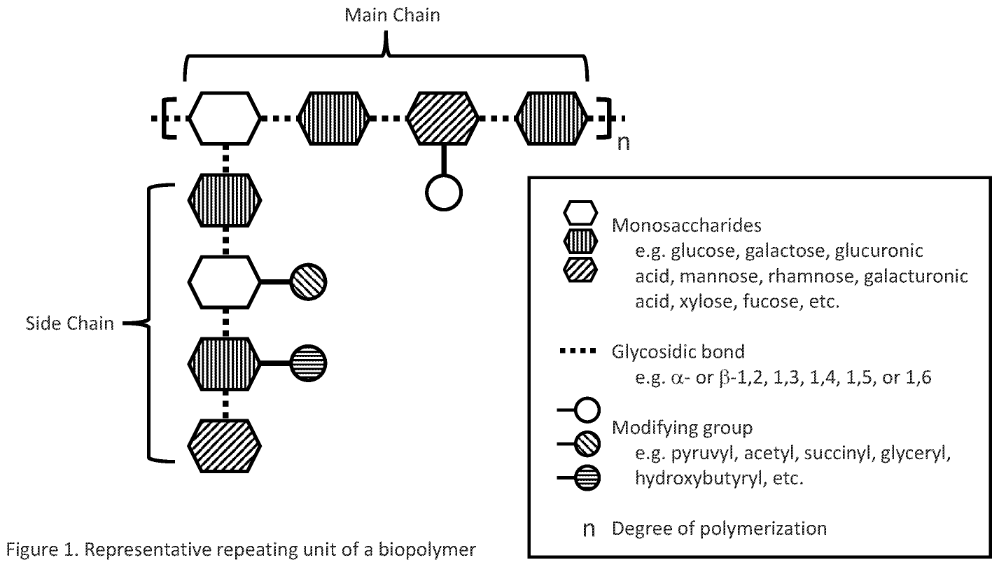

FIG. 2. Biopolymer structures. Galactoglucan is a repeating dimer of galactose and glucose, with pyruvyl and acetyl modifications. Succinoglycan is a repeating octamer of one galactose and seven glucose residues, with pyruvyl, acetyl, and succinyl modifications. Glucuronoglycan is a repeating nonamer of two galactose, two glucuronic acid, and five glucose residues with pyruvyl and acetyl modifications.

FIG. 3. Water absorption of isolated and mixed biopolymers compared to HA. The ability to bind water, as measured by mass increase, was determined for individual and mixed biopolymers. Mass increase for each biopolymer was divided by the mass increase of hyaluronic acid (HA) to derive relative fold change in water retention.

FIG. 4. Correlation between negative charge and water absorption. The charge of each biopolymer was calculated at physiological pH based on pKa of the acidic modifying group or sugar acid. This value was then divided by the number of sugar residues in a repeating unit to generate a ratio for each biopolymer. Charge density was plotted against the percent mass increase of each biopolymer.

FIG. 5. Water absorption of non-pyruvylated galactoglucan compared to HA. The ability to bind water was measured as described in Example 4 and FIG. 3. Mass increase was normalized to the value for HA, and fold change was calculated.

FIG. 6. Cytotoxicity of biopolymers. Cytotoxicity testing was performed according to the ISO 10993-5 standard. Biopolymers were dissolved in PBS at a concentration of 1% (w/v), heat pasteurized, and used in cytotoxicity assays. Values for each biopolymer were compared to both positive and negative controls.

FIG. 7. Shear thinning behavior of galactoglucan. Galactoglucan was purified according to the methods described in Example 3 and dissolved in water prior to rheological assays. To assess the shear thinning behavior of galactoglucan, flow properties of a 1% solution of galactoglucan were measured. Viscosity was determined at shear rates from 5×10\hat{ }-3 s\hat{ }-1 to 50 s\hat{ }-1 at 20° C. in a 30 mm concentric cylinder with 28 mm bob using a DHR3 rheometer (TA instruments).

### DETAILED DESCRIPTION

The present invention provides compositions and the ophthalmic or pharmaceutical use of biopolymers based on exopolysaccharides produced by non-pathogenic species of soil bacteria. Provided herein are isolated biopolymers that have substantially improved ability to treat ocular conditions, including dry eye or dry eye disease (DED).

An ocular condition is generally a disease, ailment, or condition which affects or involves the eye or one of the parts or regions of the eye.

Dry eye disease (DED) is a highly prevalent ocular surface disease. Dry eye disease can be any syndrome associated with tear film instability and dysfunction (such as increased tear evaporation and/or reduced aqueous secretion). Among the indications that are referred to by the general term “dry eye disease” are: Keratoconjunctivitis sicca (KCS), age-related dry eye, Stevens-Johnson syndrome, Sjogren's syndrome, ocular cicatrical pemphigoid, corneal injury, ocular surface infection, Riley-Day syndrome, congenital alacrima, nutritional disorders or deficiencies (including vitamin deficiencies), pharmacologic side effects, glandular and tissue destruction, autoimmune and other immunodeficient disorders, and inability to blink in comatose patients. Also included are dry eye symptoms caused by environmental exposure to airborne particulates, smoke, smog, and excessively dry air; as well as contact lens intolerance and eye stress caused by computer work or computer gaming.

There are other diseases that have a high degree of co-morbidity with dry eye disease: Allergic conjunctivitis (seasonal and chronic), blepharitis and Meibomian gland dysfunction. These conditions affect the quality and stability of the tear film, which results in dry eye signs and symptoms.

Laser assisted vision correction procedures such as photorefractive keratectomy (PRK), laser-assisted sub-epithelial keratectomy (LASEK) and laser-assisted in situ keratomileusis (LASIK) also negatively influence tear film functionality and frequently cause (temporary) dry eye disease.

Dry eye symptoms can include dryness, burning, ocular itching, ocular discomfort, photophobia, foreign body sensation, blurry vision, grittiness, scratchiness, graininess, and visual disturbance and/or loss, including blurred vision, reduced reading speed, and loss in visual acuity.

In some embodiments, the isolated biopolymers in the ophthalmic compositions are derived from Rhizobiaceae bacteria. These bacteria naturally produce low molecular weight biopolymers, which may be advantageous in some ophthalmic treatments and formulations. Manufacture of these biopolymers by fermentation is particularly advantaged relative to the processes for CMC and hyaluronic acid production. CMC is produced by treating cellulose with highly toxic chloroacetic acid as an alkylating agent, and HA derived from animals or pathogenic bacteria must be further refined to remove any zoonotic agents or toxins prior to use as an ingredient for ophthalmic care.

The present inventors have identified certain biopolymers that are efficacious for the treatment of ocular condition, including DED. The first molecule, galactoglucan, is a repeating disaccharide of galactose and glucose with pyruvyl and acetyl modifications. It may be derived from several different species of Rhizobiaceae, including the bacterium Sinorhizobium meliloti (aka Ensifer meliloti) and may be naturally produced at low molecular weight (LMW) during fermentation. It is one of two biopolymers that are naturally produced by this organism. We have demonstrated that galactoglucan has greater than 3.5× the water retention capacity of HA. A variant of galactoglucan, which lacks the pyruvyl modification, shows 1.7× the water retention capacity of HA. The second molecule, glucuronoglycan, is a repeating nonasaccharide containing galactose, glucuronic acid, and glucose with pyruvyl and acetyl modifications. It may be derived from several different species of Rhizobiaceae, including the bacterium Sinorhizobium fredi (aka Ensifer fredii) and is naturally produced as a mixture of high and low molecular weights. We have demonstrated that glucuronoglycan has 1.7× the water retention capacity of HA.

In addition to the increased water retention performance, galactoglucan and glucuronoglycan are non-toxic in a standard pre-clinical model, a prerequisite for suitability of the compounds in ophthalmic uses.

The Rhizobiaceae-derived biopolymers described herein are suitable for use in a wide range of ophthalmic care formulations, and can provide increased efficacy in the treatment of DED. Further, the production of biopolymers by fermentation, requiring little downstream processing, provides an advantaged method of production compared to incumbent technologies for the manufacture of CMC or animal- or microbial-derived HA.

### Biopolymers and Biopolymer Preparations

The Rhizobiaceae, a family of soil-dwelling, symbiotic bacteria, have been studied for decades for their ability to provide fixed nitrogen to their leguminous plant hosts, but to date have not been fully exploited as fermentative microorganisms for the production of bioindustrial, pharmaceutical, or cosmetic products. These bacteria naturally produce water-soluble exopolysaccharides, or biopolymers, which have roles in both host plant association and biofilm formation. The variety of exopolysaccharides produced by the Rhizobiaceae suggests a breadth of novel biopolymers with new functionalities that could add substantial value to several markets.

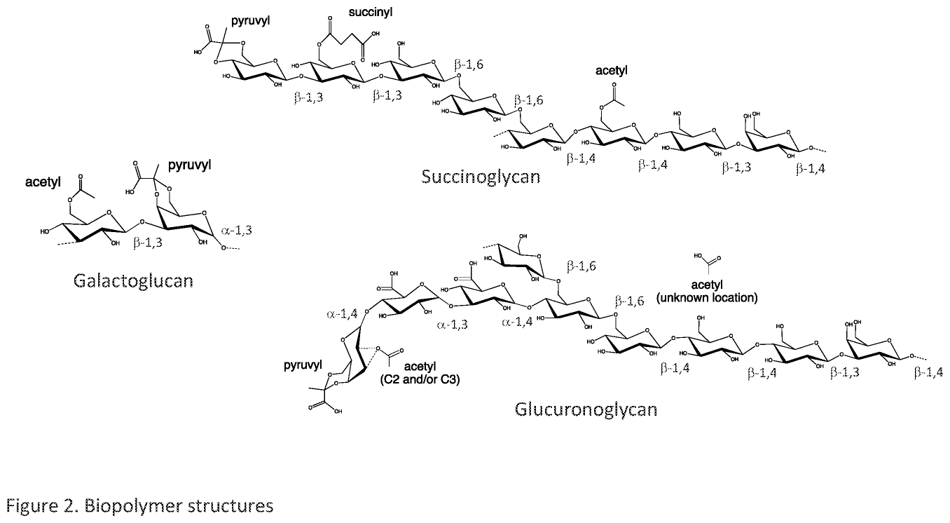

Succinoglycan and Galactoglucan Sinorhizobium (Ensifer) meliloti naturally produces two acidic exopolysaccharides: succinoglycan (EPS I), and galactoglucan (EPS II) (Barnett 2018). Succinoglycan is the major exopolysaccharide produced by S. meliloti. The repeating unit of succinoglycan (FIG. 2) consists of glucose and galactose in a 7:1 ratio with acetyl, pyruvyl and succinyl modifications (Reuber 1993). Succinoglycan is naturally produced by S. meliloti at both high and low molecular weights. The general mechanism of succinoglycan biosynthesis is relatively well understood and likely shared by related organisms that produce similar biopolymers.

Galactoglucan production is restricted to species that are phylogenetically close to S. meliloti. The repeating unit of galactoglucan (FIG. 2) consists of glucose and galactose in a 1:1 ratio with acetyl and pyruvyl modifications (Glazebrook 1989). Specific linkages are β-D-Glcp-(1-3)-α-D-Galp-(1-3), with a 6-O-acetyl on most D-glucose residues, and a 4,6-O-pyruvyl on every D-galactose (Her 1990). Galactoglucan is typically naturally produced by S. meliloti at low molecular weights. In comparison to succinoglycan, relatively little is known about the biosynthetic pathway of galactoglucan.

Glucuronoglycan Sinorhizobium (Ensifer)fredii naturally produces an acidic exopolysaccharide that consists of glucose, galactose, and glucuronic acid in a 5:2:2 ratio with acetyl and pyruvyl modifications (Djordjevic 1986) (FIG. 2). The biopolymer depicted in FIG. 2 is produced by several S.fredii type strains including NGR234, HH103 (ATCC51809), and likely USDA257 (Gray 1991, Pueppke 1999, Rodriguez-Navarro 2014). According to structural analyses, the terminal galactose on the side chain of glucuronoglycan is 4,6-pyruvylated, and it can be acetylated on either the second, third, or both carbons of the same sugar residue. A third acetyl group has also been detected in glucuronoglycan from the HH103 strain, and therefore likely the other strains as well, but its location has not been elucidated (Staehelin 2006, Rodriguez-Navarro 2014). The exo region of these S. fredii species spans 28 kb and shares a high degree of synteny with the S. meliloti cluster responsible for succinoglycan biosynthesis. Many of the genes share homology with the S. meliloti exo genes (Zhan 1990). Glucuronoglycan has a similar structure to succinoglycan, but contains glucuronic acid and is not succinylated. The main chain of glucuronoglycan consists of six sugar residues, whereas that of succinoglycan contains four. Glucuronoglycan is produced by S.fredii strains at both high and low molecular weights (Staehelin 2006).

In some embodiments, a biopolymer is provided that is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated. In some embodiments, the glucose and galactose are linked by β-1,3 glycosidic bonds and α-1,3 glycosidic bonds. In some embodiments, a biopolymer is provided that is composed of repeating disaccharide units of the structure:

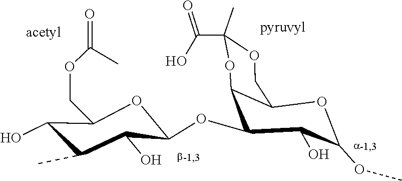

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety. In some embodiments, the biopolymer is comprised in a biopolymer preparation. In some such embodiments, the molar ratio of glucose:galactose:pyruvyl:acetyl in the biopolymer preparation is 1:1:0.4-1:0.6-1. In some embodiments, a preparation of the biopolymer is provided that comprises less than 25%, less than 20%, less than 15%, less than 10%, less than 5%, less than 3%, or less than 1% succinoglycan. In some embodiments, a preparation of the biopolymer is substantially free of succinoglycan.

In some embodiments, a biopolymer is provided that is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated. In some embodiments, the glucose and galactose are linked by β-1,3 glycosidic bonds and α-1,3 glycosidic bonds. In some embodiments, a biopolymer is provided that is composed of repeating disaccharide units of the structure:

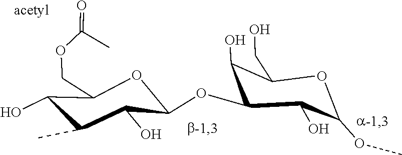

wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated. In some embodiments, the biopolymer is comprised in a biopolymer preparation. In some such embodiments, the molar ratio of glucose:galactose:pyruvyl:acetyl in the biopolymer preparation is 1:1:<0.5:0.6-1. In some embodiments, a preparation of the biopolymer is provided that comprises less than 25%, less than 20%, less than 15%, less than 10%, less than 5%, less than 3%, or less than 1% succinoglycan. In some embodiments, a preparation of the biopolymer is substantially free of succinoglycan.

In some embodiments, a biopolymer is provided, wherein the biopolymer is composed of repeating polysaccharide units of the structure:

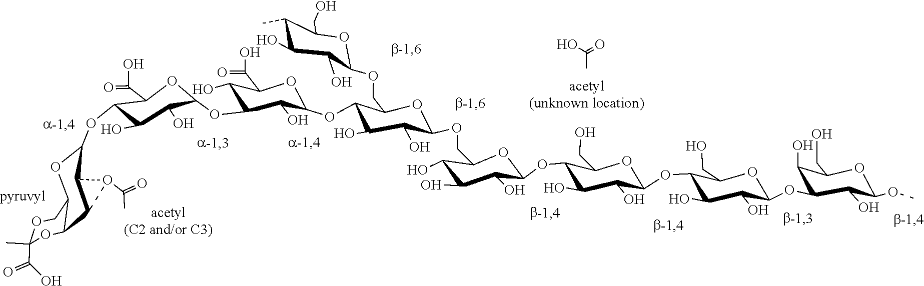

wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the disaccharide units lack the pyruvyl moiety. In some embodiments, a polysaccharide unit comprises one, two, are three acetyl groups. In some embodiments, the average number of acetyl groups per polysaccharide unit is 1-3.

In some embodiments, a biopolymer is provided, wherein the biopolymer is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45. In some embodiments, the polysaccharide unit comprises at least one galactose linked to at least one glucose. In some such embodiments, at least one galactose is linked to a glucose through a β-1,3 glycosidic bond.

In various embodiments, a composition is provided, wherein the composition comprises about 0.05% to about 5.0%, about 0.05%-about 4%, about 0.05% to about 3%, about 0.05% to about 2%, about 0.05% to about 1%, about 0.05% to about 0.5%, about 0.05% to about 0.1%, about 0.1% to about 5%, about 0.1% to about 4%, about 0.1% to about 3%, about 0.1% to about 2%, about 0.1% to about 1%, about 0.1% to about 0.5%, 0.1% to about 0.3%, about 0.5% to about 5%, about 0.5% to about 4%, about 0.5% to about 3%, about 0.5 to about 2%, or about 0.5 to about 1% of a biopolymer provided herein. In some embodiments, the composition is an ophthalmic composition. In some embodiments, the ophthalmic composition is a sterile fluid. In various embodiments, the ophthalmic composition is a sterile aqueous solution. In various embodiments, the ophthalmic composition is a sterile oil-in-water emulsion.

In various embodiments, the average molecular weight of the biopolymer in the biopolymer preparation is less than 3,000 kDa, less than 1,000 kDa, less than 300 kDa, less than 100 kDa, or less than 40 kDa. In some embodiments, the average molecular weight of the biopolymer in the biopolymer preparation is 0.5 kDa to 40 kDa or 1.6 kDa to 40 kDa.

In some embodiments, the biopolymer is capable of absorbing at least the same amount, at least 1.1-fold, at least 1.2-fold, at least 1.3-fold, at least 1.4-fold, 1.5-fold, at least 2-fold, or at least 3-fold more water than an equal amount of hyaluronic acid. In some embodiments, the biopolymer is capable of absorbing an amount of water that is at least 100%, at least 200%, at least 300%, at least 400%, or at least 500% the initial dry weight of the biopolymer preparation. Water absorption may be measured, for example, by placing a dry sample of the biopolymer or a biopolymer preparation in a humidified chamber at an approximately constant temperature for a fixed length of time. In some embodiments, the temperature is about 20° C., about 25° C., about 30° C., about 35° C., or about 37° C. In some embodiments, the fixed length of time is one day, two days, three days, four days, five days, six days, or one week.

### Strain Construction

Both S. meliloti and S. fredii are amenable to genetic modification, and a common method for strain engineering is to use homologous recombination, antibiotic resistance, and sucrose counter selection (Quandt 1993) to delete specific regions in the genome. Plasmids that contain modified genomic regions can be constructed and then used to replace native regions with targeted changes. By introduction of these non-replicating plasmids by conjugal transfer, strains with single integrations can be selected by antibiotic resistance and confirmed by PCR. Secondarily, integrated plasmids can be counter selected due the presence of the sacB gene, which encodes a levansucrase that is lethal to Gram negative bacteria in the presence of sucrose. Antibiotic sensitive, sucrose resistant strains will then either have recombined to wild type, or have incorporated a deletion, insertion, or other modification that was present in the constructed plasmid. Modified strains can be confirmed by PCR and sequencing.

Unmodified, non-domesticated strains of S. meliloti produce both succinoglycan and galactoglucan, and are suitable for the simultaneous production of both biopolymers. In certain type strains, such as Rm1021 (ATCC51124), the ability to produce galactoglucan has been lost due to lab strain domestication (Charoenpanich 2015). In the case of domesticated strains, there are several methods by which a galactoglucan producing strain can be constructed. Examples include restoration of an intact expR gene, knock out of mucR, overexpression of WggR (Bahlawane 2008), or growth in phosphate-limited medium (Mendrygal 2000).

For the production of succinoglycan in the absence of galactoglucan, the type strain Rm1021 can be used. There are several regulatory genes which can be modified resulting in strains which overproduce succinoglycan. These genes include exoR, exoS, chvl, syrM, and nodD3 (Barnett 2015). Others include syrA, mucR (Keller 1995), and exoX (Zhan 1990). If a non-domesticated strain of S. meliloti is used, it is necessary to knock out galactoglucan biosynthetic genes to generate a strain that only produces succinoglycan. The genes required for galactoglucan biosynthesis fall within a 32 kb region of pSymB and include six predicted glycosyltransferases and four genes predicted to encode proteins required for the synthesis of dTDP-glucose and dTDP-rhamnose (Becker 1997). Any of several glycosyltransferases, such as wgaB or wgeB may be excised in order to eliminate production of galactoglucan.

To produce galactoglucan in the absence of succinoglycan, wild type strains of S. meliloti with mutations in succinoglycan biosynthetic genes can be generated using pJQ200SK. Additionally, domesticated strains with the restored ability to produce galactoglucan, via any of the methods described above, may be used. The biosynthetic cluster specific for succinoglycan is located within a 22 kb region on pSymB. Structural and regulatory roles have been assigned to several of the genes in this cluster (Reuber 1993). To eliminate succinoglycan biosynthesis, any of several genes, such as exoA, exoF, exoL, exoM, exoP, exoQ, exoT, or exoY, may be excised genetically.

Glucuronoglycan is the major product of wild type S. fredii, and no modifications of strains are necessary for the production of this biopolymer. Any of the strains mentioned above may be used for production and further analysis of material.

### Methods of Making Biopolymers

For production of biopolymers, several different liquid growth media can be used. S. meliloti strains grow well on LB or TY medium, and these can be supplemented with an additional carbon source such as glucose, sucrose, or succinate to boost production of product. S. fredii can be grown on TY medium, and supplementation with additional carbon source is beneficial to production. Both S. meliloti and S. fredii can be grown on defined minimal medium, such as M9 or MOPS-mannitol, which can result in higher yields. Minimal medium allows for precise control over fermentation variables such as phosphate concentration, pH, micronutrients, sulfate concentration, and carbon source.

Alcohol precipitation may be used to purify biopolymers after fermentation. Typically, cells are removed from fermentation broth by centrifugation or filtration. High viscosity of fermentation broth may necessitate the addition of one to two volumes of water to assist in cell separation procedures. To further remove residual cells or cell debris, the cell-free supernatant may be incubated with protease. To precipitate biopolymer, isopropanol or ethanol, as well as a mono- or divalent cation such as KCl or CaCl₂ in a concentration range around 1 mM, can be added to the cell-free supernatant, typically at 1× to 2× the culture volume. Biopolymers precipitate upon mixing, and can be isolated by centrifugation or filtration. Further purification steps may be undertaken at this point to reduce salt concentrations or any cell debris that may have precipitated with the polymer. These steps may include additional alcohol washes, protease treatments, rehydration, centrifugation, dialysis, solvent washes, lyophilization, etc., that suit the desired end use. Purified product can be dried in an oven until mass stabilizes (all unbound water has evaporated). Dried product can be ground, milled, or otherwise processed to generate final, purified biopolymers.

### Ophthalmic Compositions

In various embodiments, ophthalmic compositions are provided, comprising at least one of the biopolymer(s) provided herein. In various embodiments, the ophthalmic compositions are fluid composition comprising about 0.05% to about 5.0%, about 0.05%-about 4%, about 0.05% to about 3%, about 0.05% to about 2%, about 0.05% to about 1%, about 0.05% to about 0.5%, about 0.05% to about 0.1%, about 0.1% to about 5%, about 0.1% to about 4%, about 0.1% to about 3%, about 0.1% to about 2%, about 0.1% to about 1%, about 0.1% to about 0.5%, 0.1% to about 0.3%, about 0.5% to about 5%, about 0.5% to about 4%, about 0.5% to about 3%, about 0.5 to about 2%, or about 0.5 to about 1% w/v biopolymer(s) provided herein.

“About” indicates that the item, parameter, or term so qualified encompasses a range of plus or minus ten percent above and below the value of the stated item, parameter, or term.

Common eye drop formulations contain a number of different ingredients to ensure efficacy in the treatment of ocular dysfunctions and provide correct pH and osmolarity to match typical values for human tears.

In some embodiments, the ophthalmic composition can further optionally include one or more active ingredients selected from polyethylene glycol, polyvinyl alcohol, hyaluronic acid, sodium hyaluronate, propylene glycol, carboxymethylcellulose, sodium carboxymethylcellulose, polysorbates, povidone, glycerine, and mineral oil. In some embodiments, the ophthalmic composition can further optionally include one or more inactive ingredients such as buffer, tonicity adjusting agent, antioxidant, carrier, preservative, dissolution aids, stabilizer, chelating agent, thickener, pH adjust agent, preservative, etc. Such inactive ingredients can be selected from aminomethyl propanol, boric acid, hydroxypropyl guar, preservatives, potassium chloride, purified water, sodium chloride, and sorbitol. Other optional ingredients may include calcium chloride, magnesium chloride, xanthan gum, sodium borate, sodium chlorite, erythritol, levocarnitine, carbomer copolymer type A, PURITE® (stabilized oxychloro complex), sodium borate decahydrate, citrate dihydrate, edetate disodium, octoxynol-40, polyquarternium-1, sodium lactate, cetalkonium chloride, benzalkonium chloride, glycerol, poloxamer 188, tris hydrochloride, tromethamine, or tyloxapol. These formulations are typically balanced with hydrochloric acid and/or sodium hydroxide to adjust pH.

In some embodiments, the ophthalmic composition is adjusted to have pH of 3-10, preferably pH of 5-8, more preferably pH of 6.8 to 8, even more preferably pH of 7.

In some embodiments, the ophthalmic composition further comprises hyaluronic acid. In some embodiments, the ophthalmic composition does not comprise hyaluronic acid.

In various embodiments, the ophthalmic composition can be in the form of a liquid, a solution, an emulsion, or a suspension, an ointment, a gel, a foam, an aerosol, a mist, a polymer, a film, or a paste. In various embodiments, the ophthalmic composition can be in the form of ophthalmic oil-in-water emulsions, eye hydrogels, eye drop solutions, eyebaths, eye lotions, eye inserts, eye ointments, and eye sprays and preparations, etc., preferably in the form of eye drops.

In various embodiments, the ophthalmic composition is a sterile fluid solution. In various embodiments, the ophthalmic composition is a sterile aqueous solution. In various embodiments, the ophthalmic composition is a sterile oil-in-water emulsion.

### Use of Ophthalmic Compositions

In various embodiments, methods of preventing or treating an ocular condition in a subject in need thereof are provided, comprising administering a therapeutically effective amount of ophthalmic composition comprising at least one of the biopolymer(s) provided herein to one or both eyes of the subject. A “therapeutically effective amount” of the ophthalmic composition containing the biopolymer provided herein is an amount effective to reduce the clinical signs and/or symptoms of dry eye disease (DED).

In various embodiments, the ophthalmic composition comprises about 0.01% to about 5% (w/v) of the biopolymer.

In various embodiments, the ocular condition is dry eye or dry eye disease (DED).

In various embodiments, the ophthalmic composition is administered topically to one or both eyes of the subject in need thereof. For example, the composition can be administered as one or more drops from a device for dispensing eye drops, such as an eye dropper.

In various embodiments, the method further comprises identifying a subject suffering from dry eye disease (DED) before administration of the ophthalmic composition.

In various embodiments, the subject is human or non-human animal subject. In some embodiments, the subject is a mammal. In some embodiments, the subject is a human.

Preventing or treating the ocular disease can include any one or more of the following: reducing ocular disease, preventing ocular disease, delaying the onset of ocular disease, preventing or delaying increase in severity of ocular disease, or treating ocular disease.

In various embodiments, application of the ophthalmic composition comprising the at least one biopolymer discussed herein can increase or enhance visual performance of the eye to which it is administered and help stabilize the tear film of the eye.

In some embodiments, the ophthalmic composition can be administered prophylactically, before the ocular disease, to reduce the severity of the ocular disease, to prevent ocular disease, and/or delay the onset of the ocular disease. The ophthalmic composition can also be administered therapeutically, after the onset of ocular disease, to reduce the severity of the ocular disease or to prevent the increase in severity of the ocular disease.

In some embodiments, the ophthalmic composition can result in improvement or alleviation of one or more of the following dry eye symptoms: dryness, burning, ocular itching, ocular discomfort, photophobia, foreign body sensation, blurry vision, grittiness, scratchiness, graininess, and/or visual disturbance and/or loss, including blurred vision, reduced reading speed, and loss in visual acuity.

In some embodiments, the ophthalmic composition can be administered as one or more drops one, two, three, four, five, six, seven, eight, nine, ten, or more times per day.

### EXAMPLES

### Example 1. Natural Biopolymers Produced by S. meliloti and S. fredii

Naturally occurring biopolymers produced by select Rhizobiaceae strains are shown in FIG. 2. Succinoglycan, also referred to as EPSI, is produced by several species of Rhizobiaceae including Sinorhizobium meliloti. The repeating unit consists of a linear main chain of one galactose and three glucose monosaccharides, and a side chain of four glucose molecules. Main chain sugars are linked by β-1,3 and β-1,4 glycosidic bonds. Side chain sugars are linked by β-1,3 and β-1,6 glycosidic bonds. It is acetylated on glucose three of the main chain, succinylated on glucose three of the side chain, and pyruvylated on the terminal glucose of the side chain (Reuber 1993). Galactoglucan, also referred to as EPSII, is also produced by S. meliloti and related species It is a linear, repeating dimer of galactose and glucose linked by β-1,3 and α-1,3 glycosidic bonds (Glazebrook 1989). Galactose residues are fully pyruvylated, while approximately 70% of glucose units are acetylated (Her 1990). Glucuronoglycan is produced by other species of Rhizobiaceae including Sinorhizobium fredii. It consists of a main chain composed of one galactose and five glucose monosaccharides linked by β-1,3, 0-1,4 and β-1,6 glycosidic bonds, and a side chain of two glucuronic acids and a terminal galactose linked by α-1,3, α-1,4, and β-1,4 glycosidic bonds. The terminal galactose on the side chain is both acetylated (at carbons 2 and/or 3) and 4,6-pyruvylated (Djordjevic 1986). Glucuronoglycan can be acetylated at a third, unknown location (Rodriquez-Navarro 2014).

### Example 2. Strain Construction

For targeted deletion of selected ORFs, excision of insertion elements, or correction of SNPs, a non-replicating plasmid vector with positive and negative selection markers was used in some cases to modify Rhizobiaceae bacteria. Since both S. meliloti and S. fredii are amenable to genetic modification using standard molecular biology and strain engineering techniques, this methodology allows for rapid and precise changes to their genomes to create desired genotypes. First, derivatives of the pJQ200SK plasmid (Quandt 1993) carrying deletion cassettes were generated. For deletion cassettes, regions upstream and downstream (usually 500 bp) of the target ORF including start and stop codons were amplified by PCR. For introduction of wild type DNA, regions upstream and downstream of an insertion element were amplified by PCR. Next, plasmids were assembled using the CPEC method (Quan 2009), and sequence verified prior to introduction into S. meliloti. Plasmids were introduced into S. meliloti by tri-parental mating and strains containing single integrations at homologous genomic regions were selected for antibiotic resistance, and verified by PCR using primers outside of amplified regions. Strains positive for integration of plasmids were then streaked to purification, and selected for the ability to grow on sucrose. The presence of the sacB gene on the integrated pJQ200 plasmid causes lethality when strains are grown on sucrose. Strains that are propagated on sucrose will therefore have mutations in sacB itself, or will recombine to “loop out” the integrated plasmid and either revert to wild type or harbor the deleted or modified sequence originally present in the plasmid.

Domesticated strains of S. meliloti, such as strain Rm1021, have lost the ability to produce galactoglucan (Pellock 2002), and there are several genetic modifications that can be introduced to restore this function. These modifications include deletion of the mucR gene, restoration of a wild type allele, expR101, into the expR locus (Gonzalez 1996), or introduction of a wild type expR ORF and promoter (Charoenpanich 2015). These changes all result in an Rm1021-derived strain that produces galactoglucan in addition to succinoglycan. For this study, wild type expR was introduced into strain Rm1021 using the methodology described above. This resulted in strain EXO3, which produces both S. meliloti biopolymers simultaneously.

Strain EXO3 was used to generate derivative strains that produced either succinoglycan or galactoglucan alone, by deleting ORFs that are known to be responsible for the biosynthesis of either exopolysaccharide. For example, a strain that produces succinoglycan can be generated by deletion of any of several glycosyltransferases, including wgaB, or wgeB involved in the synthesis of galactoglucan (Becker 1997). A strain that produces galactoglucan can be generated by deletion of any of several glycosyltransferases, such as exoF, exoA or exoY (Gonzalez 1996, Glazebrook 1989), involved in the initial steps of succinoglycan biosynthesis. Using the techniques described above, the wgeB ORF was deleted to generate an EXO3 derivative only capable of producing succinoglycan. To generate a galactoglucan production strain, the exoY ORF was deleted in EXO3. These strains, EX01 and EXO2 (Table 1), were used for the subsequent production of succinoglycan or galactoglucan, respectively.

The targeted deletion method described above can be used to generate strains that produce variant biopolymers, such as those that lack chemical modifications. The genes responsible for succinylation and acetylation of succinoglycan, exoH and exoZ, for example, may be deleted from the genome of S. meliloti. To generate a modified version of galactoglucan, wgaE, the gene responsible for pyruvylation was excised from S. meliloti.

TABLE 1
BioPolymer production strains.

| Strain | Species | Genotype | Product |
|---|---|---|---|
| EXO1 | S. meliloti Rm1021 | expR+, ΔwgeB | Succinoglycan |
| EXO2 | S. meliloti Rm1021 | expR+, ΔexoY | Galactoglucan |
| EXO3 | S. meliloti Rm1021 | expR+ | Succinoglycan + |
| | | | galactoglucan |
| EXO19 | S. meliloti Rm1021 | expR+, ΔexoY, | Galactoglucan |
| | | ΔwgaE | (lacking pyruvate) |
| EXO5 | S. fredii NGR234 | Wild type | Glucuronoglycan |

### Example 3. Production and Purification of Biopolymers

For bench scale growth and biopolymer production, batch cultures in shake flasks were used. Production strains were inoculated from culture plates into TY medium and grown overnight in a shaking incubator at 30° C. The next day, the overnight cultures were diluted, typically at a ratio of 1:100 or 1:200, into production medium. Production medium consisted of a defined minimal medium such as M9 containing a carbon source, either glucose or sucrose, at a concentration between 2-4% (w/v), a nitrogen source such as ammonium sulfate, a buffer to maintain neutral pH, divalent cations such as MgSO₄ and CaCl₂, trace elements, and vitamins (U.S. Pat. No. 7,371,558B2). Strains were grown in production medium for up to three days, and then harvested for purification.

Recovery and purification of biopolymers were performed by initial cell separation followed by alcohol precipitation. For high molecular weight polymers, cultures were diluted in either two or three volumes of water and supernatant was separated from cells by centrifugation. For low molecular weight biopolymers it was not necessary to dilute culture broth prior to cell separation. Approximately 1 mM CaCl₂ was then added to the supernatant, and biopolymers were precipitated at room temperature by addition of two volumes of isopropyl alcohol. Precipitates were isolated by centrifugation, and then washed in either 70% or 90% ethyl alcohol. After the wash steps, precipitates were re-isolated by low-speed centrifugation and dried overnight in a 60° C. oven until weight loss stabilized, indicating an absence of residual water. Final product was then ground using a mortar and pestle or using a bench scale mill.

### Example 4. Characterization of Biopolymers

Biopolymers are characterized by analytical methods. In some instances, NMR spectroscopy may be used to determine structural information on composition, sequence distribution, substitution pattern, and molecular weights. Biopolymers may be assayed by solution-NMR or solid-state NMR. For solution-state NMR of polysaccharides, due to the high viscosity of the material, the sample may be subjected to enzymatic digestion or pretreatment (Her et al. 1990). Samples may be assayed without pre-treatment using solid-state methods, such as ¹³C cross-polarization magic-angle spinning (CPMAS) NMR (Schaefer 1976). More detailed structural analysis and/or quantitation may be assessed by 2D NMR, for example as described in Yao 2021. The person of skill in the art understands that each analytical method has distinct advantages and disadvantages and can select an appropriate analytical method to generate desired information regarding the structure, extent of modification, and/or purity level of biopolymers. Using these methods, the extent of modification of sugars in a polysaccharide chain may be quantified. Levels of acetylation, pyruvylation, succinylation, or other modifying chemical groups, for example, may be determined for a sample of biopolymer.

### Example 5. Water Absorption

To determine the water binding capacity of biopolymers, samples were placed in a sealed, humidified chamber for five days and mass increase was measured in comparison to hyaluronic acid. Biopolymers were purified according to the procedures in Example 3. Prior to conducting the water absorption experiments, biopolymer samples were dried for 30 minutes at 60° C. to ensure that all residual water was evaporated. Small amounts (typically between 25 and 50 mg) were then weighed (value m₀) and placed into individual tared plastic or aluminum trays. All samples were placed on a platform in a sealed plastic chamber containing 250 ml of warm (approximately 35° C.) water. The entire chamber containing all samples was then placed into an incubator at 30° C. After five days, the humidified chamber was opened, and individual samples were weighed (value m) to calculate mass increase. Water binding capacity (WBC) for each sample was calculated according to the following equation: (m−m₀)/m₀. This raw value represents the degree of swelling and can be expressed as percent mass increase by multiplying by 100.

A pure, 5 kDa preparation of hyaluronic acid (HAworks) was used as a control for the water binding experiments. WBC, measured as described above, of hyaluronic acid was typically between 200 and 300%. Higher molecular weights of HA (100 kDa HAworks, and >1000 kDa Acros Organics) were tested and showed similar water absorbing capacities as the low molecular weight sample. To calculate fold change of experimental samples, WBC values for mass increase of biopolymers were normalized to the WBC values for HA within an experiment.

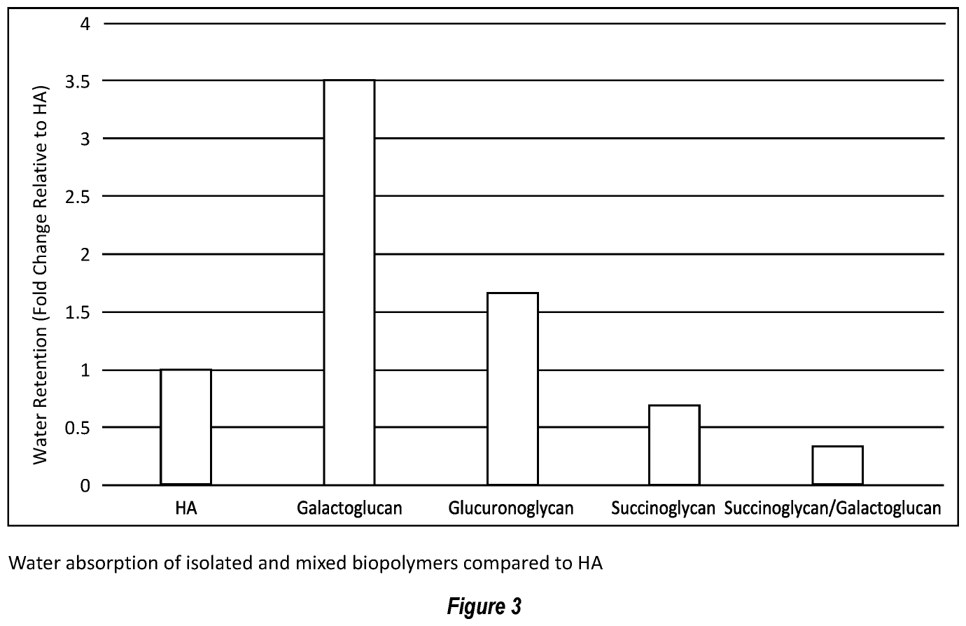

FIG. 3 shows that the WBC of isolated galactoglucan is increased by as much as 3.5-fold in comparison to HA. This WBC value was replicable across multiple experiments. The raw percent increase in mass was 650% for galactoglucan, which was substantially higher than the 185% increase measured for HA. After further incubation in the humidified chamber, the mass increase for galactoglucan reached as high as 720% of its initial mass. Results were similar when galactoglucan was derived from multiple carbon sources including glucose, sucrose, and corn syrup. Glucuronoglycan, isolated from S. fredii, showed a 1.7-fold increase in water binding relative to HA. The raw value for percent mass increase for glucuronoglycan was 311%. Isolated succinoglycan, in contrast to galactoglucan and glucuronoglycan, displayed decreased water binding in comparison to HA (0.7-fold decrease or 129% raw mass increase), and a mixture of succinoglycan and galactoglucan was reduced (0.3-fold decrease or 60% raw mass increase) even further. In the case of galactoglucan, the improved WBC was only observed when galactoglucan was purified independently, and not as the naturally occurring mixture of both galactoglucan and succinoglycan from S. meliloti.

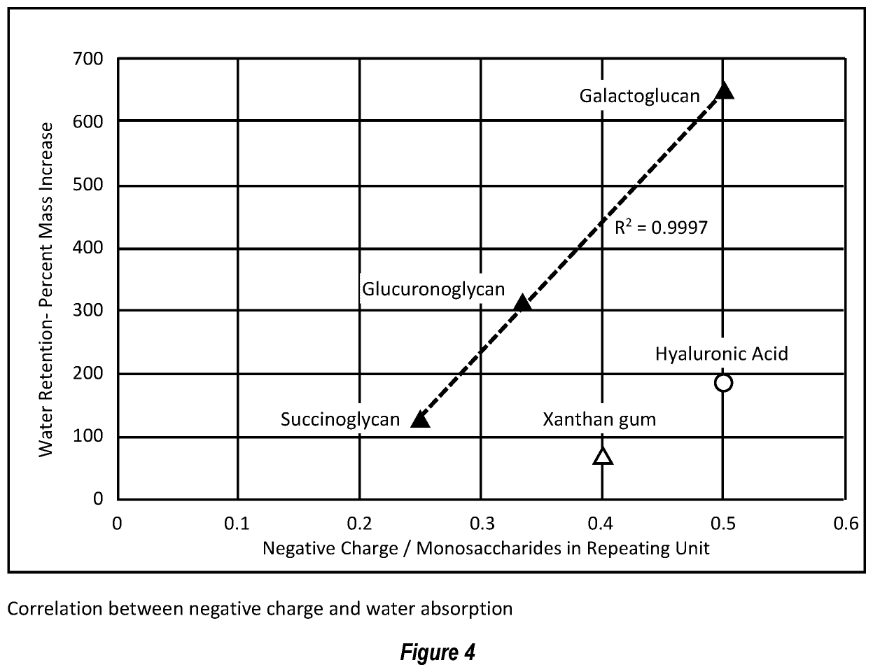

Although monosaccharide type and content, chemical modifications, glycosidic linkages, and molecular weight may all affect the behavior of a biopolymer, for the biopolymers derived from these species of Rhizobiaceae, the degree of negative charge appears to be a predominant factor in WBC. FIG. 4 shows that for the S. meliloti and S. fredii biopolymers, there is a correlation between negative charge and water binding performance. For values on the x-axis, negative charge for each repeating unit was calculated based on the pKa of chemical groups or sugar acids at physiological pH. For example, the two glucuronic acids and the pyruvate modification of glucuronoglycan each contribute one negative charge at neutral pH, and therefore the ratio of negative charge to total sugars in the repeating unit is 1:3, or 0.33. For galactoglucan, this ratio is 1:2, and for succinoglycan this ratio is 1:4. These ratios were plotted against the values for percent mass increase of each biopolymer. As shown in FIG. 4, galactoglucan, the molecule with the highest ratio of negative charge to monosaccharides in the repeating unit had the highest capacity for water binding. Other rhizobial biopolymers fit precisely upon this trendline with a high R² value. Hyaluronic acid (gray circle), which also has a charge to monosaccharide ratio of 1:2, did not fit on the trendline, indicating that for this molecule, something other than or in addition to charge ratio affects water retention. Xanthan gum (Modernist Pantry) displayed poor water binding capacity in comparison to HA, and also did not fit on the trendline.

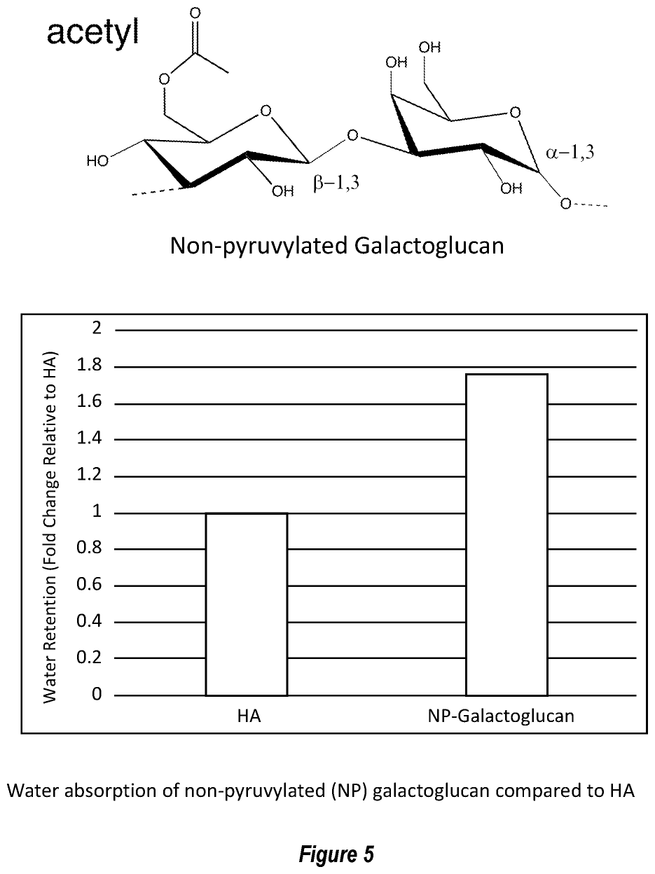

FIG. 5 shows the structure of a non-pyruvylated galactoglucan molecule (NP-galactoglucan), derived from an S. meliloti strain with the wgaE gene excised. The NP-galactoglucan molecule also retained more water than the HA control, although not to the extent of the fully pyruvylated galactoglucan. Compared to HA, the NP-galactoglucan molecule displayed a 1.7-fold increase in the ability to bind water.

### Example 6. Cytotoxicity

To test for cytotoxicity, biopolymers were purified according to Example 3 and resuspended in a Ca-, Mg-free solution of PBS at a concentration of 1% (w/v). These solutions were then heat pasteurized for 30 minutes at 60° C. in a water bath. The cytotoxicity assay described below was carried out at Pacific Biolabs in Hercules, CA.

Test Procedure: A sterile filter paper with a flat surface measuring 1.0 cm2 total surface area was saturated with ˜0.1 mL of the test solution and placed directly on the cell culture monolayer in the center of a 10 cm2 well. Triplicate preparations were prepared. Triplicate positive and negative controls were tested in the same manner as the test articles. All wells were incubated for not less than 24 hours at 37±1° C. in a humidified incubator with 5±1% CO2. After incubation, the test articles and controls were gently removed from the wells. The cell cultures were examined under an inverted microscope with 100× magnification for cytotoxic response. The response was graded on a scale of 0-4. The achievement of a numerical grade greater than 2 is considered a cytotoxic effect.

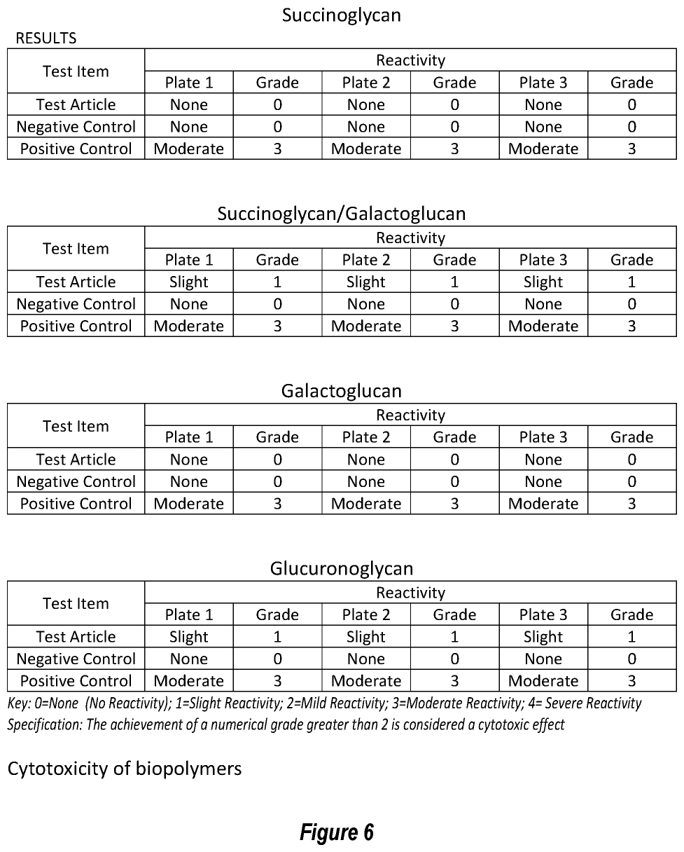

This study was conducted according to ISO 10993-5:2009. A value of 0 is considered no reactivity, and a value of 1 is considered only slightly reactive. FIG. 6 shows that isolated succinoglycan, galactoglucan, glucuronoglycan, and a mixture of succinoglycan and galactoglucan are not cytotoxic according to this assay.

### Example 7. Formulations for Ophthalmic Care

Biopolymers and/or derivatives thereof are produced and purified according to Example 3. Biopolymers are dissolved in phosphate buffered saline (PBS) at final concentrations between 0.1-2% (w/v). Prior to use, solutions are sterilized either by filtration or heat pasteurization.

### Example 8. Dry Eye Testing

The formulations described in Example 7 are used in studies to examine the effect on dry eye. Several volunteers above the age of 18 are instructed to apply PBS solution containing biopolymer (the test solution) to their right eye 5 times per day over the course of 30 days. The volunteers are also instructed to apply PBS solution with no biopolymer (the control solution) to their left eye at the same frequency. At the beginning of the study baseline measurements are taken using the TearLab® Osmolarity System, according to manufacturer's recommended protocols, and the Schirmer test. For the Schirmer test, test strips (Sports World Vision) are applied between the bottom eyelid and the eye for five minutes, after which point the migration distance on the strip is measured. During the trial period, volunteers are retested after 5, 10, and 30 days to measure osmolarity and Schirmer values.

### Example 9. Shear Thinning Behavior of Galactoglucan

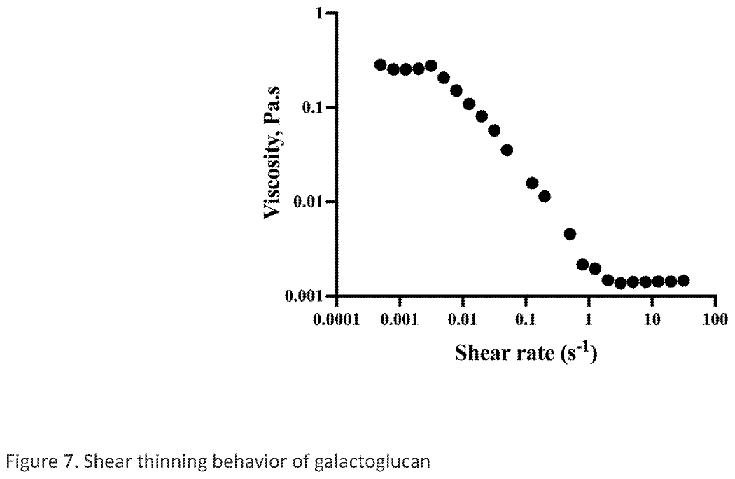

The behavior of galactoglucan was observed using a rheometer to measure shear thinning properties. Shear thinning behavior is a desired property for functional ingredients in artificial tear formulations, since viscosity of shear thinning materials decreases during the blink cycle. This property helps maintain a viscous coating over the ocular surface and effectively disperse biopolymer during blinking. Galactoglucan was purified according to the methods described in Example 3 and dissolved in water prior to rheological assays. To assess the shear thinning behavior of galactoglucan, flow properties of a 1% solution of galactoglucan were measured. Viscosity was determined at shear rates from 5×10\hat{ }-3 s\hat{ }-1 to 50 s\hat{ }-1 at 20° C. in a 30 mm concentric cylinder with 28 mm bob using a DHR3 rheometer (TA instruments). FIG. 7 shows that as shear rate increases, viscosity of galactoglucan decreases. This behavior is typical for pseudoplastic, shear thinning materials. FIG. 7 also shows that galactoglucan has inherently low native viscosity (approx. 0.2 Pa.s) at low shear rates, another property that is desirable for functional artificial tear ingredients.

We claim:

1. An ophthalmic composition comprising a biopolymer preparation comprising a biopolymer that is composed of repeating disaccharide units comprising glucose and galactose, wherein at least 60%, at least 65%, at least 70%, at least 75%, at least 80%, at least 85%, or at least 90% of the glucose is acetylated, and wherein at least 40%, at least 45%, at least 50%, at least 55%, at least 60%, at least 65%, at least 70%, at least 75%, at least 80%, at least 85%, at least 90%, at least 95%, or at least 99% of the galactose is pyruvylated.

2. The ophthalmic composition of claim 1, wherein the glucose and galactose are linked by β-1,3 glycosidic bonds and α-1,3 glycosidic bonds.

3. An ophthalmic composition comprising a biopolymer preparation comprising a biopolymer that is composed of repeating disaccharide units of the structure: 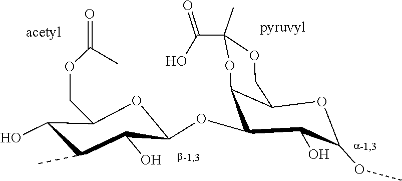
wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 60%, no more than 55%, no more than 50%, no more than 45%, no more than 40%, no more than 35%, no more than 30%, no more than 25%, no more than 20%, no more than 15%, no more than 10%, no more than 5%, or no more than 1% of the disaccharide units lack the pyruvyl moiety.

4. The ophthalmic composition of any one of claims 1-3, wherein the molar ratio of glucose:galactose:pyruvyl:acetyl in the biopolymer is 1:1:0.4-1:0.6-1.

5. The ophthalmic composition of any one of claims 1-4, wherein the average molecular weight of the biopolymer in the biopolymer preparation is less than 3,000 kDa, less than 1,000 kDa, less than 300 kDa, less than 100 kDa, or less than 40 kDa.

6. The ophthalmic composition of any one of claims 1-4, wherein the average molecular weight of the biopolymer in the biopolymer preparation is 0.5 kDa to 40 kDa.

7. The ophthalmic composition of any one of the preceding claims, wherein the ophthalmic composition comprises about 0.05 to about 5% w/v biopolymer.

8. The ophthalmic composition of any one of claims 1-6, wherein the ophthalmic composition comprises about 0.5 to about 2% w/v biopolymer.

9. The ophthalmic composition of any one of the preceding claims, wherein the biopolymer preparation is capable of absorbing at least the same amount, at least 1.5-fold, at least 2-fold, or at least 3-fold more water than an equal amount of hyaluronic acid.

10. The ophthalmic composition of any one of the preceding claims, wherein the biopolymer preparation is capable of absorbing an amount of water that is at least 100%, at least 200%, at least 300%, at least 400%, or at least 500% the initial dry weight of the biopolymer preparation.

11. The ophthalmic composition of claim 9 or claim 10, wherein water absorption is measured by placing a dry sample of the biopolymer preparation in a humidified chamber at 30° C. for five days.

12. The ophthalmic composition of any one of the preceding claims, wherein the biopolymer preparation is less than 25%, less than 20%, less than 15%, less than 10%, less than 5%, less than 3%, or less than 1% succinoglycan, or is substantially free of succinoglycan.

13. The ophthalmic composition of any one of the preceding claims, wherein the ophthalmic composition is a sterile fluid.

14. An ophthalmic composition comprising a biopolymer preparation comprising a biopolymer that is composed of repeating disaccharide units comprising glucose and galactose, wherein at least 60%, at least 65%, at least 70%, at least 75%, at least 80%, at least 85%, or at least 90% of the glucose is acetylated, and wherein less than 15%, less than 10%, or less than 5% of the galactose is pyruvylated.

15. The ophthalmic composition of claim 14, wherein the glucose and galactose are linked by β-1,3 glycosidic bonds and α-1,3 glycosidic bonds.

16. An ophthalmic composition comprising a biopolymer preparation, wherein the biopolymer is composed of repeating disaccharide units of the structure: 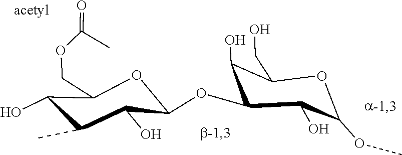
wherein the dotted lines represent the bonds between disaccharide units; wherein no more than 40%, no more than 35%, no more than 30%, no more than 25%, no more than 20%, no more than 15%, no more than 10%, or no more than 5% of the disaccharide units lack the acetyl moiety; and wherein no more than 15%, no more than 10%, or no more than 5%, of the disaccharide units are pyruvylated.

17. The ophthalmic composition of any one of claims 14-16, wherein the molar ratio of glucose:galactose:pyruvyl:acetyl in the biopolymer is 1:1:<0.5:0.6-1.

18. The ophthalmic composition of any one of claims 14-17, wherein the average molecular weight of the biopolymer in the biopolymer preparation is less than 3,000 kDa, less than 1,000 kDa, less than 300 kDa, less than 100 kDa, or less than 40 kDa.

19. The ophthalmic composition of any one of claims 14-17, wherein the average molecular weight of the biopolymer in the biopolymer preparation is 0.5 kDa to 40 kDa.

20. The ophthalmic composition of any one of claims 14-19, wherein the ophthalmic composition comprises about 0.05 to about 5% w/v biopolymer.

21. The ophthalmic composition of any one of claims 14-19, wherein the ophthalmic composition comprises about 0.5 to about 2% w/v biopolymer.

22. The ophthalmic composition of any one of claims 14-21, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.

23. The ophthalmic composition of any one of claims 14-22, wherein the biopolymer preparation is capable absorbing an amount of water that is at least 100%, at least 200%, at least 300%, or at least 400% the initial dry weight of the biopolymer preparation.

24. The ophthalmic composition of claim 22 or claim 23, wherein water absorption is measured by placing a dry sample of the biopolymer preparation in a humidified chamber at 30° C. for five days.

25. The ophthalmic composition of any one of claims 14-24, wherein the biopolymer preparation is less than 25%, less than 20%, less than 15%, less than 10%, less than 5%, less than 3%, or less than 1% succinoglycan.

26. The ophthalmic composition of any one of claims 14-25, wherein the ophthalmic composition is a sterile fluid.

27. An ophthalmic composition comprising a biopolymer preparation comprising a biopolymer that is composed of repeating polysaccharide units of the structure: 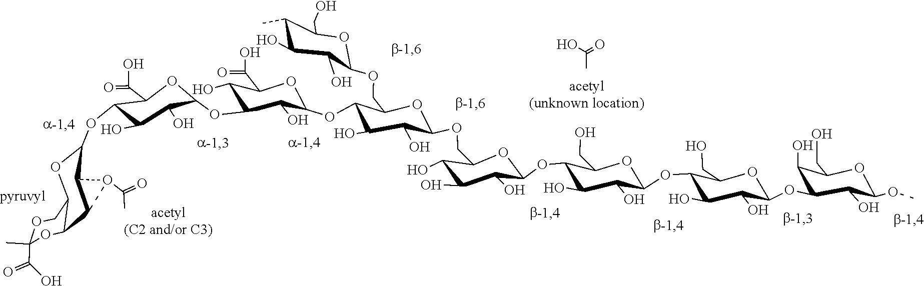
wherein the dotted lines represent the bonds between polysaccharide units; wherein no more than 30%, no more than 25%, no more than 20%, no more than 15%, no more than 10%, no more than 5%, or no more than 1% of the polysaccharide units lack an acetyl moiety; and wherein no more than 10%, no more than 5%, or no more than 1% of the polysaccharide units lack the pyruvyl moiety.

28. The ophthalmic composition of claim 27, wherein the average molecular weight of the biopolymer in the biopolymer preparation is less than 3,000 kDa, less than 1,000 kDa, less than 300 kDa, less than 100 kDa, or less than 40 kDa.

29. The ophthalmic composition of claim 27, wherein the average molecular weight of the biopolymer in the biopolymer preparation is 1.6 kDa to 40 kDa.

30. The ophthalmic composition of any one of claims 27-29, wherein the ophthalmic composition comprises about 0.05 to about 5% w/v biopolymer.

31. The ophthalmic composition of any one of claims 27-29, wherein the ophthalmic composition comprises about 0.5 to about 2% w/v biopolymer.

32. The ophthalmic composition of any one of claims 27-31, wherein the biopolymer preparation is capable of absorbing at least the same amount, at least 1.1-fold, at least 1.2-fold, at least 1.3-fold, at least 1.4-fold, or at least 1.5-fold more water than an equal amount of hyaluronic acid.

33. The ophthalmic composition of any one of claims 27-32, wherein the biopolymer preparation is capable of absorbing an amount of water that is at least 100%, at least 200%, at least 300%, or at least 400% the initial dry weight of the biopolymer preparation.

34. The ophthalmic composition of claim 32 or claim 33, wherein water absorption is measured by placing a dry sample of the biopolymer preparation in a humidified chamber at 30° C. for five days.

35. The ophthalmic composition of any one of claims 27-33, wherein the ophthalmic composition is a sterile fluid.

36. An ophthalmic composition comprising a biopolymer preparation comprising a biopolymer that is composed of repeating polysaccharide units, wherein each polysaccharide unit comprises 2-15 or 2-12 or 2-10 monosaccharides, and wherein the biopolymer has a negative charge:monosaccharide ratio in the repeating polysaccharide unit of at least 0.3, or at least 0.35, or at least 0.4, or at least 0.45.

37. The ophthalmic composition of claim 36, wherein the polysaccharide unit comprises at least one galactose linked to at least one glucose.

38. The ophthalmic composition of claim 37, wherein at least one galactose is linked to a glucose through a β-1,3 glycosidic bond.

39. The ophthalmic composition of any one of claims 36-38, wherein the average molecular weight of the biopolymer in the biopolymer preparation is less than 3,000 kDa, less than 1,000 kDa, less than 300 kDa, less than 100 kDa, or less than 40 kDa.

40. The ophthalmic composition of any one of claims 36-38, wherein the average molecular weight of the biopolymer in the biopolymer preparation is 0.5 kDa to 40 kDa.

41. The ophthalmic composition of any one of claims 36-40, wherein the ophthalmic composition comprises about 0.05 to about 5% w/v biopolymer.

42. The ophthalmic composition of any one of claims 36-40, wherein the ophthalmic composition comprises about 0.5 to about 2% w/v biopolymer.

43. The ophthalmic composition of any one of claims 36-42, wherein the biopolymer preparation is capable of absorbing at least the same amount, at least 1.1-fold, at least 1.5-fold, at least 2-fold, or at least 3-fold more water than an equal amount of hyaluronic acid.

44. The ophthalmic composition of any one of claims 36-43, wherein the biopolymer preparation is capable of absorbing an amount of water that is at least 100%, at least 200%, at least 300%, or at least 400% the initial dry weight of the biopolymer preparation.

45. The ophthalmic composition of claim 43 or claim 44, wherein water absorption is measured by placing a dry sample of the biopolymer preparation in a humidified chamber at 30° C. for five days.

46. The ophthalmic composition of any one of claims 36-45, wherein the ophthalmic composition is a sterile fluid.

47. The ophthalmic composition of any one of the preceding claims, wherein the composition comprises hyaluronic acid.

48. The ophthalmic composition of any one of claims 1-46, wherein the composition does not comprise hyaluronic acid.

49. The ophthalmic composition of any one of the preceding claims, wherein the composition is a fluid.

50. The ophthalmic composition of any one of the preceding claims, wherein the ophthalmic composition at least 70%, at least 80%, at least 85%, at least 90%, or at least 95% water.

51. A method of treating or preventing an ocular condition in a subject, comprising administering a therapeutically effective amount of the ophthalmic composition of any one of the preceding claims to the eye of the subject.

52. The method of claim 51, wherein the ocular condition is dry eye disease (DED).

53. The method of claim 51 or claim 52, where the ophthalmic composition is an ophthalmic oil-in-water emulsion, eye hydrogel, eye drop solution, eyebath, eye lotion, eye insert, eye ointment, eye foam, or eye spray.

54. The method of any one of claims 51-53, wherein the ophthalmic composition is an eye drop solution.

55. The method of any one of claims 51-54, wherein the ophthalmic composition is administered prophylactically.

56. The method any one of claims 51-55, wherein the ophthalmic composition is administered therapeutically after the onset of ocular disease.

57. The method of any one of claims 51-56, wherein the ophthalmic composition results in improvement or alleviation of one or more symptoms selected from dryness, burning, ocular itching, ocular discomfort, photophobia, foreign body sensation, blurry vision, grittiness, scratchiness, graininess, and visual disturbance and/or loss, including blurred vision, reduced reading speed, and loss in visual acuity.
